(19) United States
(12) Patent Application Publication
(10) Pub. No.: US 2016/0310058 A1
(43) Pub. Date: Oct. 27, 2016

(54) METHOD AND SYSTEM FOR SENSING EMOTIONAL DATA OF USERS

(71) Applicant: FIH (HONG KONG) LIMITED, Kowloon (HK)

(72) Inventor: CHENG-HSUN LI, New Taipei (TW)

(21) Appl. No.: 14/721,635

(22) Filed: May 26, 2015

(30) Foreign Application Priority Data
Apr. 24, 2015 (TW) 104113140

(51) Int. Cl. A61B 5/16; A61B 5/00

(52) U.S. Cl. CPC A61B 5/165; A61B 5/6801; A61B 5/725; A61B 2560/0475

(57) ABSTRACT

An electronic device includes a storage device, at least one processor, an indication device, a wearable sensor, and one or more modules that are stored in the storage device and executed by the at least one processor. The one or more modules includes a time setting module, a spectrum processing, a counting module, and a determination module. The time setting module controls the wearable sensor to sense physiological parameters of users and output a spectrum signal corresponding to the physiological parameters. The spectrum processing module filters the spectrum signal. The counting module counts a number of times the spectrum signal passes through the spectrum processing module and thus the counting module outputs an increment-by-one signal. The determination module has a predetermined threshold and determines whether the increment-by-one signal exceeds the predetermined threshold, the determination module activates the indication device if the increment-by-one signal exceeds the predetermined threshold.

### FIELD

The subject matter herein generally relates to emotional state sensing system, and more particularly to a system and a method for sensing physiological parameters of users.

### BACKGROUND

Emotions play an important role in interpersonal interactions. It is important to detect and respond to both positive and negative emotional changes. Positive aspects of a work environment may stimulate productive emotional changes in individuals. Emotions such as joy nay lead to higher levels of productivity and efficiency.

### BRIEF DESCRIPTION OF THE DRAWINGS

Implementations of the present technology will now be described, by way of example only, with reference to the attached figures.

FIG. 1 is a block diagram of an electronic device employing an emotional state sensing system, according to an exemplary embodiment.

FIG. 2 is a flowchart of one embodiment of an emotional state sensing method using the emotional state sensing system of FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising,” when utilized, means “including, but not necessarily limited to”; it specifically indicates open-ended inclusion or membership in the so-described combination, group, series and the like.

The present disclosure is described in relation to an emotional state sensing system and an emotional state sensing method using the same.

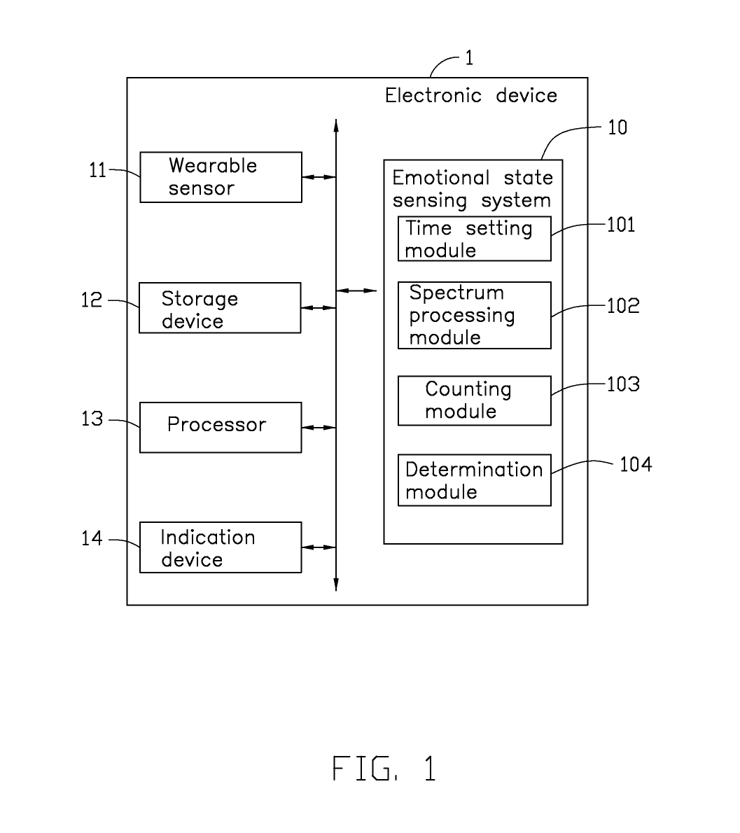

FIG. 1 illustrates an embodiment of an electronic device 1 including an emotional state sensing system 10, according to an exemplary embodiment. The electronic device 1 may be a cell phone, a smart watch, a personal digital assistant, a tablet computer, or any other computing devices.

The electronic device 1 further includes a wearable sensor 11, a storage device 12 providing one or more memory functions, at least one processor 13, and an indication device 14.

The wearable sensor 11 is configured to sense one or more physiological parameters of users. In at least one embodiment, the wearable sensor 11 is a heart rate sensor and is used to obtain physiological parameters such as heart rate and heartbeat strength. Generally, the physiological parameters is associated with emotional states (e.g., tired, anxious or hurried) of the users. For example, an increased heart rate may represent changes of the emotional states. In addition, the wearable sensor 11 converts the physiological parameters into a spectrum signal via fast fourier transform (FFT), and the spectrum signal is stored in the storage device 12 and processed by the processor 13. In other embodiments, the wearable sensor 11 can be a respiration sensor or an electro dermal activity sensor.

In at least one embodiment, the emotional state sensing system 10 may include computerized instructions in the form of one or more programs, which are stored in the storage device 12 and executed by the processor 13 to perform operations of the electronic device 1. The storage device 12 stores one or more programs, such as programs of the operating system, other applications of the electronic device 1, and various kinds of data, such as animated visual images. In some embodiments, the storage device 12 may include a memory of the electronic device 1 and/or an external storage card, such as a memory stick, a smart media card, a compact flash card, or any other type of memory card. FIG. 1 illustrates only one example of the electronic device 1 that may include more or fewer components than as illustrated, or have a different configuration of the various components. The processor 13 can be a microcontroller. The indication device 14 is electronically coupled to the processor 13 and is configured to output an indication signal to notify the users. In at least one embodiment, the indication device 14 can be a loudspeaker. In other embodiments, the indication device 14 can be a vibrator.

In at least one embodiment, the emotional state sensing system 10 may include one or more modules, for example, a time setting module 101, a spectrum processing module 102, a counting module 103, and a determination module 104. In general, the word “module”, as used herein, refers to logic embodied in hardware or firmware, or to a collection of software instructions, written in a programming language, such as, Java, C, or assembly. One or more software instructions in the modules may be embedded in firmware, such as in an EPROM. The modules described herein may be implemented as either software and/or hardware modules and may be stored in any type of non-transitory computer-readable medium or other storage device. Some non-limiting examples of non-transitory computer-readable medium include CDs, DVDs, BLU-RAY, flash memory, and hard disk drives.

The time setting module 101 is configured to set a time range. At a period of the time range, the time setting module sends a first control command to the wearable sensor 11 for turning on the wearable sensor 11. In at least one embodiment, the time range can be about 8:00-12:00 and 13:00-17:00. That is, the wearable sensor 11 can be turned off beyond the time range for conserving power.

The spectrum processing module 102 is configured to filter the spectrum signal stored in the storage device 12. In detail, the spectrum processing module 102 has a predetermined frequency band, and a range of the predetermined frequency band is adjustable and incompletely covers all spectrum signals output from the wearable sensor 11 for providing a suitable window size to perform useful spectrum analysis. Optionally, the spectrum processing module 102 is a high pass filter (HPF). When the spectrum signal is greater than the predetermined frequency band, the spectrum signal can pass through the spectrum processing module 102. Since the spectrum signal output from the wearable sensor 11 is stored in the storage device 12 in real time, thus, the spectrum processing module 102 can continue to process a plurality of spectrum signals to improve efficiency and accuracy.

The counting module 103 is configured to count when the spectrum signal passes through the spectrum processing module 102, thus, the counting module 103 outputs an increment-by-one signal, and the initial value of the increment-by-one signal is set to 0. If the spectrum signal cannot pass through the spectrum processing module 102, the counting module 103 is inactivated.

The determination module 104 has a predetermined threshold and is configured to determine whether the increment-by-one signal exceeds the predetermined threshold at a period of time (such as 10 seconds). If the increment-by-one signal exceeds the predetermined threshold at the period of time, the determination module 104 activates the indication device 14. If the increment-by-one signal does not exceed the predetermined threshold at the period of time, the determination module 104 deactivates the indication device 14.

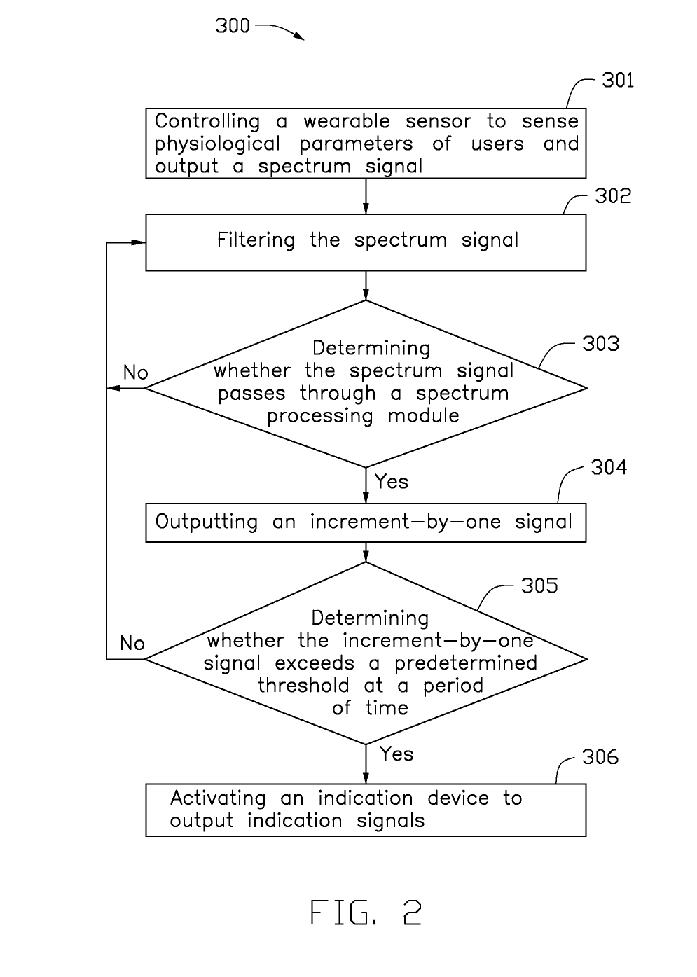

FIG. 2 illustrates a flowchart of an example emotional state sensing method 300 of the disclosure. The emotional state sensing method 300 is provided by way of example, as there are a variety of ways to carry out the emotional state sensing method 300. The emotional state sensing method 300 described below can be carried out using the functional units of the emotional state sensing system 10 as illustrated in FIG. 1, for example, and various elements of this figure are referenced in explaining the example emotional state sensing method 300. Each block shown in FIG. 2 represents one or more processes, methods, or subroutines which are carried out in the example emotional state sensing method 300. Furthermore, the order of blocks is illustrative only and the order of the blocks can change. Additional blocks can be added or fewer blocks may be utilized without departing from the scope of this disclosure. The example emotional state sensing method 300 can begin at block 301.

At block 301, the time setting module sets a time range, and then controls a wearable sensor to be turned on. Thus, the wearable sensor senses one or more physiological parameters of users and consequently outputs a spectrum signal.

At block 302, the spectrum processing module filters the spectrum signal.

At block 303, determining whether the spectrum signal passes through the spectrum processing module. If the spectrum signal passes through the spectrum processing module, block S304 is implemented. If the spectrum signal cannot pass through the spectrum processing module, the counting module is inactivated and the procedure turns back to block S302.

At block 304, the counting module outputs an increment-by-one signal.

At block 305, determining whether the increment-by-one signal exceeds a predetermined threshold at a period of time. If the increment-by-one signal exceeds the predetermined threshold at the period of time, block S306 is implemented. If the increment-by-one signal does not exceed the predetermined threshold at the period of time, the procedure turns back to block S302.

At block 306, the determination module activates the indication device to output indication signals.

In other embodiments, the emotional state sensing system 10 can be applied in two separately electronic devices. For example, the time setting module 101 is stored in a first electronic device (such as a smart wristband) having the wearable sensor 11. The spectrum processing module 102, the counting module 103, and the determination module 104 are stored in a second electronic device (such as a mobile phone) having the storage device 12, the at least one processor 13, and the indication device 14. Optionally, the first electronic device communicates with the second electronic device via wireless communications to send the spectrum signal to the second electronic device.

In summary, the time setting module 101 sets the time range, and then controls the wearable sensor 11 to sense physiological parameters of users. The spectrum processing module 102 filters the spectrum signal to allow the spectrum signal greater than the predetermined frequency band to pass through the spectrum processing module 102. The counting module 103 outputs the increment-by-one signal, and the determination module 104 activates the indication device 14 when the increment-by-one signal exceeds the predetermined threshold. Thus, the emotional state sensing system 10 is capable of effectively detecting and responding to both positive and negative emotional changes, and the feedback of the indication device 14 may be formulated to change the user's emotional state.

The embodiments shown and described above are only examples. Many details are often found in the art such as the other features of the emotional state sensing system and the emotional state sensing method using the same. Therefore, many such details are neither shown nor described. Even though numerous characteristics and advantages of the present technology have been set forth in the foregoing description, together with details of the structure and function of the present disclosure, the disclosure is illustrative only, and changes may be made in the details, especially in matters of shape, size and arrangement of the parts within the principles of the present disclosure up to, and including the full extent established by the broad general meaning of the terms used in the claims. It will therefore be appreciated that the embodiments described above may be modified within the scope of the claims.

What is claimed is:

1. An electronic device comprising:
a storage device;
at least one processor;
an indication device coupled to the processor and configured to output an indication signal; and
a wearable sensor coupled to the one processor,
wherein, a time setting module is stored on the storage device and is executable by the processor and the time setting module is configured to control the wearable sensor to sense physiological parameters of users and output a spectrum signal corresponding to the physiological parameters;
wherein, a spectrum processing module is stored on the storage device and is executable by the processor and the spectrum processing module is configured to filter the spectrum signal;
wherein, a counting module is stored on the storage device and is executable by the processor and the counting module is configured to count a number of times the spectrum signal passes through the spectrum processing module and output an increment-by-one signal; and
wherein, a determination module is stored on the storage device and is executable by the processor and the determination module is configured to determine whether the increment-by-one signal exceeds a predetermined threshold and activate the indication device in event the increment-by-one signal exceeds the threshold.

2. The electronic device as claimed in claim 1, wherein the time setting module sets a time range, and controls the wearable sensor to sense the physiological parameters at a period of the time range.

3. The electronic device as claimed in claim 1, wherein the spectrum processing module has a predetermined frequency band, when the spectrum signal is greater than the predetermined frequency band, the spectrum signal passes through the spectrum processing module.

4. The electronic device as claimed in claim 1, wherein the spectrum processing module is a high pass filter (HPF).

5. The electronic device as claimed in claim 1, further comprising a first electronic device and a second electronic device communicating with the first electronic device, the time setting module is stored in the first electronic device having the wearable sensor, the spectrum processing module, the counting module, and the determination module is stored in the second electronic device having the storage device, the at least one processor, and the indication device.

6. A computer-implemented method for emotional state sensing using an electronic device, the electronic device comprising an indication device and a wearable sensor, the method comprising execution of steps comprising:
controlling, by a time setting module, the wearable sensor to sense physiological parameters of users and output a spectrum signal corresponding to the physiological parameters;
filtering, by a spectrum processing module, the spectrum signal;
determining, by a counting module, whether the spectrum signal passes through the spectrum processing module;
outputting, by a counting module, an increment-by-one signal, if the spectrum signal passes through the spectrum processing module;
determining, by a determination module, whether the increment-by-one signal exceeds a predetermined threshold; and
activating, by the determination module, the indication device if the increment-by-one signal exceeds the predetermined threshold.

7. The method as claimed in claim 6, further comprising setting a time range by the time setting module and controlling the wearable sensor to sense the physiological parameters at a period of the time range.

8. The method as claimed in claim 6, wherein the spectrum processing module has a predetermined frequency band, when the spectrum signal is greater than the predetermined frequency band, the spectrum signal passes through the spectrum processing module.

9. The method as claimed in claim 6, wherein if the spectrum signal does not pass through the spectrum processing module, the counting module is inactivated and the procedure turns back to the step of filtering, by a spectrum processing module, the spectrum signal.

10. The method as claimed in claim 6, wherein if the increment-by-one signal does not exceed the predetermined threshold, the procedure turns back to the step of filtering, by a spectrum processing module, the spectrum signal.

11. A non-transitory storage medium having stored instructions that, when executed by a processor of an electronic device, causes the electronic device to perform a method for emotional state sensing, the electronic device comprising an indication device and a wearable sensor, the method comprising:
controlling, by a time setting module, the wearable sensor to sense physiological parameters of users and output a spectrum signal corresponding to the physiological parameters;
filtering, by a spectrum processing module, the spectrum signal;
determining, by a counting module, whether the spectrum signal passes through the spectrum processing module;
outputting, by a counting module, an increment-by-one signal, if the spectrum signal passes through the spectrum processing module;
determining, by a determination module, whether the increment-by-one signal exceeds a predetermined threshold at a period of time; and
activating, by the determination module, the indication device if the increment-by-one signal exceeds the predetermined threshold at the period of time.

12. The non-transitory storage medium as claimed in claim 11, further comprising setting a time range by the time setting module and controlling the wearable sensor to sense the physiological parameters at a period of the time range.

13. The non-transitory storage medium as claimed in claim 11, wherein the spectrum processing module has a predetermined frequency band, when the spectrum signal is greater than the predetermined frequency band, the spectrum signal passes through the spectrum processing module.

14. The non-transitory storage medium as claimed in claim 11, wherein if the spectrum signal does not pass through the spectrum processing module, the counting module is inactivated and the procedure turns back to the step of filtering, by a spectrum processing module, the spectrum signal.

15. The non-transitory storage medium as claimed in claim 11, wherein if the increment-by-one signal does not exceed the predetermined threshold at the period of time, the procedure turns back to the step of filtering, by a spectrum processing module, the spectrum signal.
